(19) United States
(12) Patent Application Publication
(10) Pub. No.: US 2015/0320927 A1
(43) Pub. Date: Nov. 12, 2015

(54) Split Tip Catheter for Dialysis Treatment

(71) Applicant: Mahase Nardeo, Oaks, PA (US)

(72) Inventor: Mahase Nardeo, Oaks, PA (US)

(73) Assignee: VESCUTECH MEDICAL, LLC, Oaks, PA (US)

(21) Appl. No.: 14/803,178

(22) Filed: Jul. 20, 2015

Related U.S. Application Data
(60) Provisional application No. 62/141,306, filed on Apr. 1, 2015.

(51) Int. Cl. A61M 1/36; A61M 25/00

(52) U.S. Cl. CPC A61M 1/3661; A61M 25/003; A61M 25/0032; A61M 25/0028; A61M 2025/0031; A61M 2025/0034; A61M 2025/0037; A61M 2025/0098; A61M 2202/0413; A61M 2202/0021; A61M 2207/00; A61M 25/0009

(57) ABSTRACT

Disclosed is a split tip catheter having a tapered venous lumen and a tapered arterial lumen, wherein each of the lumens includes a D-shaped cross section. In one embodiment, the length of the venous lumen is greater than the length of the arterial lumen. The venous lumen and the arterial lumen are secured in a side-by-side configuration via an outer sleeve such that the proximal end of the outer sleeve is aligned with the proximal ends of the venous lumen and the arterial lumen, leaving the distal ends of the venous lumen and the arterial lumen free. The proximal ends of the outer sleeve, the venous lumen, and the arterial lumen are insert molded to a hub to connect the lumens to extensions such as venous and arterial blood lines for dialysis treatments.

### CROSS-REFERENCE TO RELATED APPLICATIONS

This application claims the benefit of U.S. Provisional Patent Application No. 62/141,306, filed Apr. 1, 2015, which is hereby incorporated by reference in its entirety.

### FIELD OF THE INVENTION

The present invention relates generally to the field of medical devices. More particularly, the present invention is directed to a split tip catheter for dialysis treatment.

### BACKGROUND OF THE INVENTION

Split tip catheters are widely used for dialysis treatment. These catheters generally include a first lumen and a second lumen, wherein the two lumens are secured together in a side-by-side configuration. One known method of manufacturing two lumens in a side-by-side configuration is by extruding two integral lumens and then splitting a septum therebetween, allowing the lumens to separate. The splitting is a delicate process, however, and one that is difficult to achieve during manufacturing. If a portion of one or both of the lumens is unintentionally cut during the splitting process, the lumens can leak during use.

Another known method of manufacturing two lumens in a side-by-side configuration is to attach them together via adhesives or the like. Once bonded, a hub is molded at the proximal ends of the lumens, connecting the proximal extensions and allowing blood lines to be connected to the arterial and the venous ports for the purpose of dialyzing patients with kidney failure.

During the molding process, however, excess stress is induced on the lumens adjacent to the hub. In many cases, excess stress during the molding process also causes the lumens to leak during use. Leaky lumens can be highly problematic and risky for patients. For instance, leaky lumens cannot effectively filter harmful wastes, salt, and excess fluid from the patients' blood. Thus, there is a need in the prior art for an improved split tip dialysis catheter and a method of manufacture of producing the same, whereby the method prevents the catheter from leaking.

The present invention provides a split tip catheter comprising a venous lumen and an arterial lumen. Each of the lumens comprises a semi-circle or a D-shaped cross section, such that the two lumens can be secured together via an outer sleeve to form a substantially circular cross section having a diameter. The outer sleeve is further connected to an insert-molded hub having a venous line and an arterial line therein. In this way, the venous lumen and the arterial lumen are in fluid communication with the venous line and the arterial line attached to the proximal ends thereof, respectively.

The present invention is advantageous in that the outer sleeve provides added protection against environmental solvents used to clean the exit site after use. Additionally, the durometer of the outer sleeve may be lower than the durometer of the lumens to prevent kinking during use while keeping the lumens together and allowing for back tunneling for better placement of the distal ends.

### SUMMARY OF THE INVENTION

In view of the disadvantages inherent in the known types of catheters now present in the prior art, the present invention provides an improved split tip catheter that is used during dialysis treatment.

In one embodiment, the present catheter comprises a tapered venous lumen and a tapered arterial lumen, wherein each of the lumens comprises a substantially D-shaped cross section. In this way, the venous lumen and the arterial lumen can be assembled together to form a substantially circular cross section having a diameter. The distances between the proximal end and the distal end of the venous lumen and the arterial lumen define the their respective lengths. Preferably, the length of the venous lumen is greater than the length of the arterial lumen.

The venous lumen and the arterial lumen are secured together via a cylindrical outer sleeve. Preferably, the proximal ends of the lumens are aligned so that the distal end of the venous lumen extends beyond the distal end of the arterial lumen. Additionally, the outer sleeve is biased toward the proximal ends of the lumens, leaving the distal ends of the venous lumen and the arterial lumen detached. In use, the proximal ends of the lumens are connected to venous and arterial blood lines for dialysis treatments, whereby the venous and arterial blood lines are secured within a hub that is insert molded to the outer sleeve.

It is therefore an object of the present invention to provide a split tip catheter comprising a venous lumen and an arterial lumen, each of the lumens comprising a D-shaped cross section.

It is another object of the present invention to provide a split tip catheter having a venous lumen and an arterial lumen, wherein the venous lumen is longer than the arterial lumen.

It is still another object of the present invention to provide a split tip catheter having a venous lumen and an arterial lumen, wherein the venous lumen and the arterial lumen are held together via an outer sleeve.

It is still another object of the present invention to provide a split tip catheter having an outer sleeve with a lower durometer than that of a venous lumen and an arterial lumen to prevent kinking.

It is still another object of the present invention to provide a split tip catheter that comprises multiple apertures disposed on distal ends of the lumens.

A final object of the present invention is to provide a split tip catheter that may be readily fabricated from materials that permit relative economy and commensurate with durability.

In the light of the foregoing, these and other objects are accomplished in accordance of the principles of the present invention, wherein the novelty of the present invention will become apparent from the following detailed description and appended claims.

### BRIEF DESCRIPTION OF THE DRAWINGS

The above and other objects and advantages of the present invention will be apparent upon consideration of the following detailed description, taken in conjunction with the accompanying exemplary drawings, in which like reference characters refer to like parts throughout, and in which:

FIG. 1A shows a side perspective view of a D-shaped venous lumen.

FIG. 1B shows a side perspective view of a D-shaped arterial lumen.

FIG. 1C shows a perspective view of an outer sleeve.

FIG. 1D shows a side perspective view of another embodiment of the D-shaped lumen of the present invention.

FIG. 2 shows a perspective view of the present invention in an assembled configuration.

FIG. 3 shows a side elevational view of the present invention in operation.

### DETAILED DESCRIPTION OF THE INVENTION

The present invention is directed towards a catheter that comprises a split tip. For purposes of clarity, and not by way of limitation, illustrative views of the present split tip catheter are described with references made to the above-identified figures. Various modifications obvious to one skilled in the art are deemed to be within the spirit and scope of the present invention.

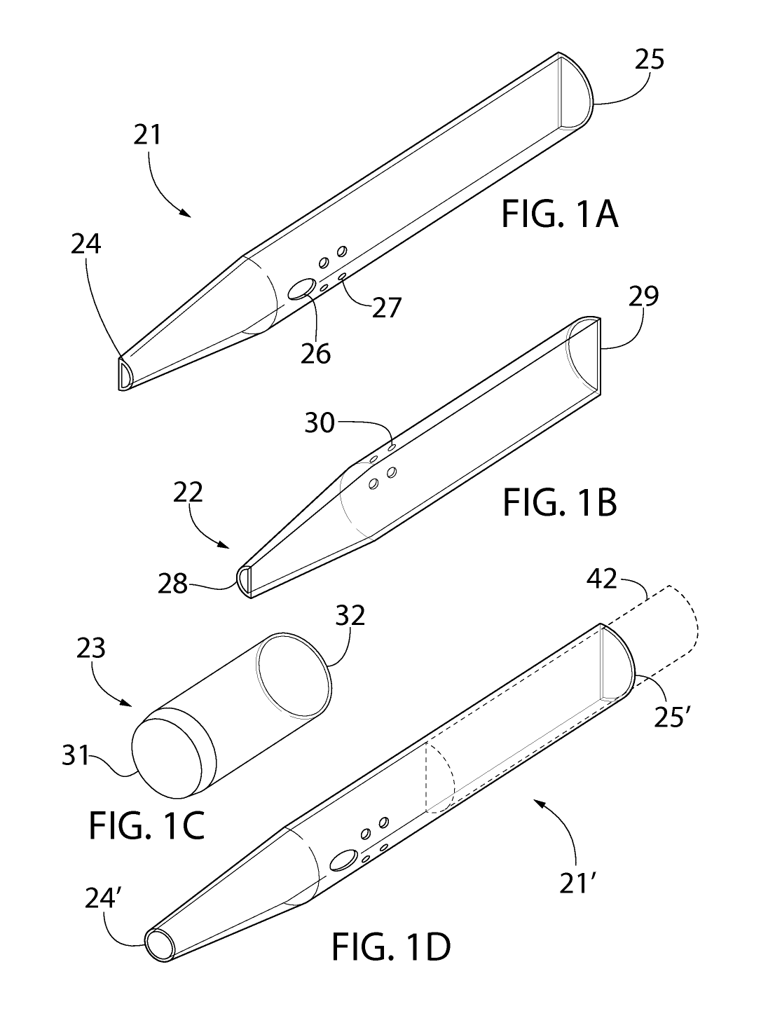

Referring now to FIG. 1A, there is shown a perspective view of a venous lumen of the present invention. The venous lumen 21 comprises an elongated tubular shape having a distal end 24 and a proximal end 25, wherein each of the distal end 24 and the proximal end 25 is open. The distance between the distal end 24 and the proximal end 25 defines a length of the venous lumen 21. The cross section of the venous lumen 21 is substantially D-shaped or semi-circular. In this regard, the venous lumen 21 comprises a curved side and a flat side.

The diameter of the cross section of the venous lumen 21 is generally constant over its length, but it decreases towards the distal end 24 such that the distal end 24 is tapered. In this regard, the flat side of the venous lumen 21 remains planar while the curved side of the venous lumen 21 slopes toward the flat side near the distal end 24. It is contemplated that approximately less than one-third of the length of the venous lumen 21 is tapered, depending upon embodiment.

The venous lumen 21 further comprises a plurality of apertures thereon, wherein the apertures are biased toward the distal end 24 thereof. The apertures facilitate fluid flow and prevent blockages during dialysis. The apertures are disposed on the curved side of the venous lumen 21, and short of the portion of the curved side that tapers. In the illustrated embodiment, the venous lumen 21 comprises a first set of apertures 26 and a second set of apertures 27, wherein each of the apertures 26, 27 comprises a substantially circular shape with a defined diameter. The diameter of the first set of apertures 26 vary from the diameter of the second set of apertures 27 such that the first set of apertures 26 and the second set of apertures 27 are different in size.

Referring now to FIG. 1B, there is shown a perspective view of an arterial lumen of the present invention. The arterial lumen 22 comprises an elongated tubular shape having a distal end 28 and a proximal end 29, wherein each of the distal end 28 and the proximal end 29 is open. The distance between the distal end 28 and the proximal end 29 defines a length of the arterial lumen 22. The length of the arterial lumen 22 is less than the length of the venous lumen. The cross section of the arterial lumen 22 is substantially D-shaped or semi-circular, so that the arterial lumen 22 comprises a curved side and a flat side. It is contemplated that the cross section of the arterial lumen 22 is substantially equal in shape and dimension as the cross section of the venous lumen.

The diameter of the cross section of the arterial lumen 22 is generally constant over its length, but it decreases towards the distal end 28 such that the distal end 28 is tapered. In this regard, the flat side of the arterial lumen 22 remains planar while the curved side of the arterial lumen 22 slopes towards the flat side. It is contemplated that approximately less than one-third of the length of the arterial lumen 22 is tapered, depending upon embodiment.

The arterial lumen 22 further comprises a plurality of apertures 30 thereon, wherein the apertures 30 are biased toward the distal end 28 thereof. Similar to the apertures disposed on the venous lumen, the apertures 30 prevent blockages from occurring while the present catheter is in use. The apertures 30 are disposed on the curved side of the arterial lumen 22. Preferably, the apertures 30 are not disposed on the sloped portion of the curved side of the arterial lumen 22.

In some embodiments, the lumens comprise a rounded distal end, as shown in FIG. 1D. The lumen 21′ comprises a substantially D-shaped cross section having a flat side and a rounded side. The lumen 21′ comprises a venous lumen and/or an arterial lumen. The distal end 24′ of the lumen 21′ comprises an open rounded cross section so that it is substantially circular or conical. Accordingly, while it is contemplated that some embodiments of the present lumen may comprise a circular cross section having a tubular structure, the preferred embodiment of the lumens comprise a D-shaped cross section so that it has a smaller profile. The illustrated embodiment is advantageous in that it can be used with an obturator 42. In the illustrated embodiment, the obturator 42 also comprises a substantially D-shaped cross section. The obturator 42 is configured so that it can fit within the interior of the lumen 21′ when inserted from the proximal end 25′ thereof.

The present invention further comprises an outer sleeve, as shown in FIG. 1C. The outer sleeve 23 is substantially cylindrical in shape and includes an open distal end 31, an open proximal end 32, and a hollow interior. The distance between the distal end 31 and the proximal end 32 of the outer sleeve 23 defines a length of the outer sleeve 23. The length of the outer sleeve 23 is less than the length of the arterial lumen, and thus less than the length of the venous lumen. The outer sleeve 23 comprises a circular cross section having a diameter. The diameter is generally uniform over the length of the outer sleeve 23, but the distal end 31 is preferably trimmed so as to form a knifepoint around the edge thereof. The outer sleeve 23 is composed of a substantially flexible material, and in some embodiments, a transparent material so as to allow a user to view the interior of the outer sleeve 23.

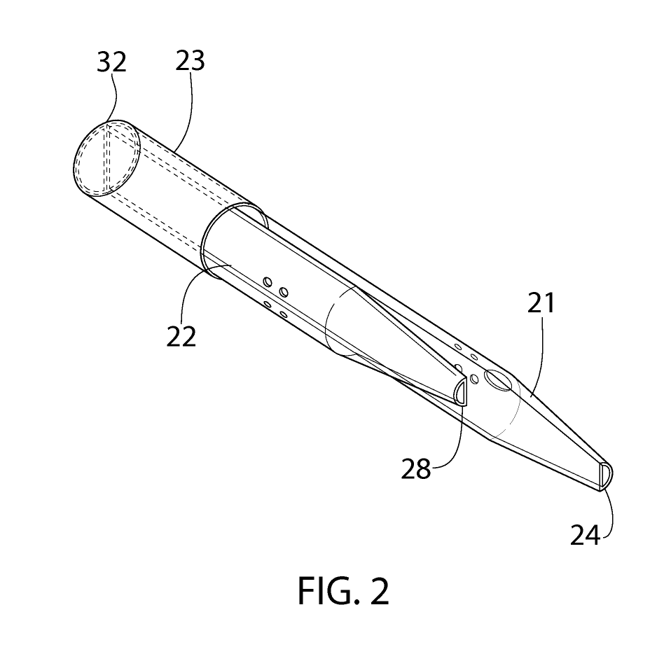

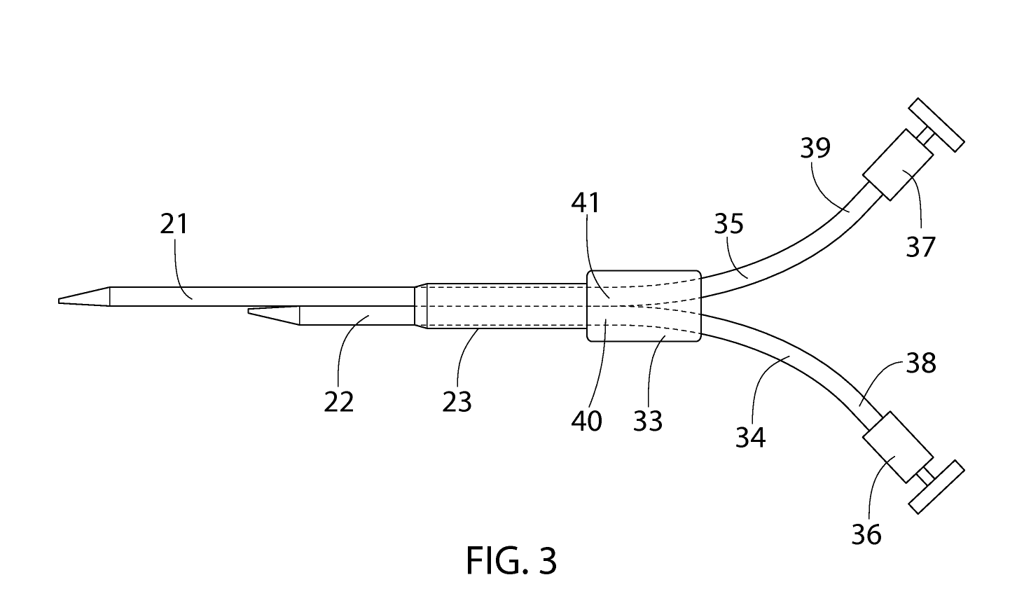

Referring now to FIGS. 2 and 3, there is shown a perspective view of the present invention in an assembled configuration and a view of the present invention in operation, respectively. Because each of the venous lumen 21 and the arterial lumen 22 comprises a D-shaped or a semi-circular cross section, the flat sides of the lumens 21, 22 can be assembled together in a side-by-side configuration to form a substantially circular cross section having a diameter.

The venous lumen 21 and the arterial lumen 22 are secured together via the outer sleeve 23. The diameter of the cross section of the outer sleeve 23 is slightly greater than the diameter of the venous lumen 21 and the arterial lumen 22 assembled together. In this way, outer sleeve 23 can snugly fit over the venous lumen 21 and the arterial lumen 22, securing them together in place, as depicted in FIG. 2. Additionally, the distal end 31 of the outer sleeve 23 is trimmed so as to create a smooth transition from the outer surfaces of the lumens to the outer surface of the outer sleeve 23. It is contemplated that the outer sleeve 23 is composed of a material having a low durometer so as to prevent the lumens 21, 22 from kinking during use while keeping the lumens together and allowing for back tunneling for better placement of the distal ends of the lumens 21, 22.

Preferably, the proximal ends of the lumens 21, 22 are flush against the proximal end 32 of the outer sleeve 23 so as to create a single proximal end, while the distal ends of the venous lumen and the arterial lumen remain detached. Additionally, the distal end 24 of the venous lumen 21 extends beyond the distal end 28 of the arterial lumen 22. In operation, the proximal end of each of the outer sleeve 23, the venous lumen 21 and the arterial lumen 22 is insert molded to a hub 33 to connect the lumens 21, 22 to extensions such as blood lines. Thus, the lumens 21, 22 may establish fluid communication with blood lines during dialysis treatments. It is contemplated that the insert molding process does not exert excessive stress on the lumens 21, 22.

The hub 33 is substantially hollow cylindrical in shape and comprises an open first end opposite an open second end, wherein the first end is attached to the proximal ends of the lumens 21, 22 and the outer sleeve 23. The diameter of the opening of the first end of the hub 33 is less than or substantially equal to the diameter of the cross section of the outer sleeve 23 so that the outer sleeve 23 and the hub 33 create a seal when attached.

Additionally, the first end of the hub 33 comprises a venous blood line 35 and an arterial blood line 34 connected thereto. Because the hub 33 comprises a hollow interior, the venous blood line 35 and the arterial blood line 34 can move freely therein. In the illustrated embodiment, the proximal end of the venous lumen 21 and the proximal end of the arterial lumen 22 connect to a venous blood line 35 and an arterial blood line 34, respectively. The lumens 21, 22 can connect to the blood lines 34, 35 via resealable seal. The proximal end 38 of the arterial blood line 34 and the proximal end 39 of the venous blood line 35 are connected to a dialysis machine via luer connectors 36, 37.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. A split tip catheter for dialysis treatment, comprising:
a venous lumen having an open proximal end and an open tapered distal end, wherein a distance between said proximal end of said venous lumen and said distal end of said venous lumen defines a length of said venous lumen;
an arterial lumen having an open proximal end and an open tapered distal end, wherein a distance between said proximal end of said arterial lumen and said distal end of said arterial lumen defines a length of said arterial lumen;
said length of said arterial lumen less than said length of said venous lumen;
a cylindrical outer sleeve having an open distal end and an open proximal end;
said outer sleeve securing said venous lumen and said arterial lumen together such that said proximal end of said venous lumen, said proximal end of said arterial lumen, and said proximal end of said outer sleeve are aligned, creating a single proximal end;
said single proximal end in fluid communication with a venous blood line and an arterial blood line disposed within a hub.

2. The split tip catheter of claim 1, wherein each of said venous lumen and said arterial lumen comprises a plurality of apertures biased toward said distal ends thereof.

3. The split tip catheter of claim 2, wherein said plurality of apertures varies in size.

4. The split tip catheter of claim 1, wherein each of said venous lumen and said arterial lumen comprises a flat side and a curved side, forming a D-shaped cross section.

5. The split tip catheter of claim 4, wherein said flat side of said venous lumen directly contacts said flat side of said arterial lumen.

6. The split tip catheter of claim 1, wherein said distal end of said venous lumen extends beyond said distal end of said arterial lumen.

7. The split tip catheter of claim 1, wherein said venous blood line and said arterial blood line can move freely within said hub.

8. The split tip catheter of claim 1, wherein said distal end of said venous lumen and said distal end of said arterial lumen are detached.

9. The split catheter of claim 1, wherein said single proximal end is insert molded to said hub.

10. A split tip catheter for dialysis treatment, comprising:
a venous lumen having an open proximal end and an open tapered distal end;
an arterial lumen having an open proximal end and an open tapered distal end;
said arterial lumen shorter than said venous lumen;
each of said venous lumen and said arterial lumen having a D-shaped cross section;
a cylindrical outer sleeve having an open distal end and an open proximal end;
said outer sleeve securing said venous lumen and said arterial lumen together such that said proximal end of said venous lumen, said proximal end of said arterial lumen, and said proximal end of said outer sleeve are aligned, creating a single proximal end;
a cylindrical hub having a first end and a second end;
said first end of said hub connected to said single proximal end;
a venous blood line having a proximal end and a distal end;
an arterial blood line having a proximal end and a distal end;
each of said distal end of said venous blood line and said distal end of said arterial blood line connected to said first end of said hub.

11. The split tip catheter of claim 10, wherein each of said venous lumen and said arterial lumen comprises a plurality of apertures biased toward said distal ends thereof.

12. The split tip catheter of claim 11, wherein said plurality of apertures varies in size.

13. The split tip catheter of claim 10, wherein said distal end of said venous lumen extends beyond said distal end of said arterial lumen.

14. The split tip catheter of claim 10, wherein said venous blood line and said arterial blood line can move freely within said hub.

15. The split tip catheter of claim 10, wherein said distal end of said venous lumen and said distal end of said arterial lumen is detached.

16. The split catheter of claim 10, wherein said single proximal end is insert molded to said hub.

17. The split catheter of claim 10, further comprising a luer connector on each of said proximal end of said venous blood line and said proximal end of said arterial blood line.
